(19) United States
(12) Patent Application Publication
(10) Pub. No.: US 2009/0163950 A1
(43) Pub. Date: Jun. 25, 2009

(54) Surgical needle holder with attached suture cutter

(76) Inventor: Peter J. Waldman, Morrisville, VT (US)

Correspondence Address: BENJAMIN APPELBAUM, PH.D.;ATTORNEY AT LAW, 27 BENNINGTON DRIVE, FLANDERS, NJ, 07836, US

(21) Appl. No.: 12/004,212

(22) Filed: Dec. 20, 2007

(51) Int. Cl. A61B 17/062

(52) U.S. Cl. 606/222

(57) ABSTRACT

A surgical instrument comprises two elongated arms terminating in a finger ring and a jaw for holding a needle. The arms are pivotably fastened to each other and a scissors member that includes a finger ring and a cutting edge proximate the scissors member second end. The arms can be locked together by a pair of interengageable clasps having a plurality of projections, which interlock when the clasps are engaged. A stop member attached to one arm limits the scissors member's movement. When the device is in a closed position, a needle is retained between the jaws, the needle being attached to a length of suture material. The device is used to place the suture, tie the suture, and the scissors member is opened, and then used to cut the untied suture material after the suture has been tied.

### FIELD OF THE INVENTION

Embodiments of the present invention include an instrument for holding a surgical needle, and which instrument includes a suture cutting mechanism so that the user can place, tie and cut a suture using a single instrument.

### BACKGROUND OF THE INVENTION

In clinical settings such as a hospital emergency room, clinician's office, or the like, lacerations or similar types of wounds, or surgical incisions, require closure. In the case of a wound, first bleeding is stopped, the site “cleaned up”, and after appropriate treatment of the wound, the wound is closed, frequently by means of one or more sutures. Sutures are generally packaged as a sterile kit, containing the combination of a needle attached to a particular length of suture material, for example, a length ranging from 12 to 18 inches. The particular type of needle and suture material vary, and are selected based on the particular application. Generally, when a clinician, such as, for example only, an emergency physician, family doctor, general or plastic surgeon, occupational medicine physician or urgent care physician does suturing in an office or emergency room setting, no assistant is present to cut the sutures, necessitating the following steps for each suture placed:

placing the suture and tying it using the needle holder;

putting down the needle holder;

picking up the scissor;

cutting the suture;

putting down the scissor; and

picking up the needle holder for the next suture, and repeating the process until the length of suture material is used, or the wound is closed.

Several instruments which perform the combined function of a surgical needle holder and suture cutter have been described in the prior art, examples of which are described below.

Gmeiner, in U.S. Pat. No. 2,315,326 discloses a device that incorporates a third arm as the scissor, but the scissor arm is incorporated atop one of the members of the needle holder. This device requires the surgeon to release a latching mechanism to free the scissor arm in order to use it, in what appears to be a cumbersome action.

U.S. Pat. No. 4,452,246 discloses a device wherein the cutting device is at the proximal portion of the grasping end of the instrument, so that it appears to be difficult to see the cutting edges.

U.S. Pat. No. 5,439,471 discloses the suture-cutting mechanism as part of the clasping end of the needle holder, at the distal end. One potential problem with this device is that one could easily cut the suture material inadvertently while using this instrument for tying knots, which is another function of this device.

The present invention is a combination surgical needle holder and scissor, which can be used, for example only, during laceration repair in an ambulatory care setting. With the present invention, the redundant steps described above for a typical suturing procedure are eliminated, and the clinician can simply place, tie and cut the sutures using a single instrument. This not only saves time, but also decreases the risk of dropping an instrument; eliminates the need for a suture-cutting scissors in a typical laceration tray; and decreases the risk of accidental exposure to blood-borne pathogens through the picking up and putting down of instruments, as well as reduces risk of infection to the patient.

The present invention represents an improvement over the prior art devices. In an embodiment of the present invention, the scissor function is initiated by using the incorporated finger loop where the user's index finger is placed. This does not require any additional steps or change in the position of the hand, as the typical position for holding a needle holder is with the thumb and middle fingers in the loops of the needle holder and the index finger resting adjacent to the middle finger, guiding the middle finger. Using the inventive instrument, the user's index finger is in the same position, but now inside the finger ring of the instrument's scissor arm. The inventive instrument can be used by either right or left handed persons by flipping the instrument over 180 degrees.

### BRIEF SUMMARY OF THE INVENTION

It is an object of the present invention to provide a single device that can hold a suture needle for placing and tying a suture, and which is capable of cutting the suture.

Another object of the present invention is to provide a combination suture needle holder and scissors that is sterilizable.

Still another object of the present invention is to provide a combination suture needle holder and scissors that can be included as part of a suturing kit.

Yet another object of the present invention is to provide a combination suture needle holder and scissors that is durable, yet economical enough to be considered disposable.

A surgical instrument comprises two elongated arms terminating in a finger ring and a jaw for holding a needle. The arms are pivotably fastened to each other and a scissors member that includes a finger ring and a cutting edge proximate the scissors member second end. The arms can be locked together by a pair of interengageable clasps having a plurality of projections, which interlock when the clasps are engaged. A stop member attached to one arm limits the scissors member's movement. When the device is in a closed position, a needle is retained between the jaws, the needle being attached to a length of suture material. The device is used to place the suture, tie the suture, and the scissors member is opened, and then used to cut the untied suture material after the suture has been tied.

### BRIEF DESCRIPTION OF THE SEVERAL VIEWS OF THE DRAWING

FIG. 1 is a plan view of an embodiment of the present invention in the closed position.

FIG. 2 illustrates the embodiment of FIG. 1 in an open position.

FIG. 3 illustrates the scissors member.

### DETAILED DESCRIPTION OF THE INVENTION

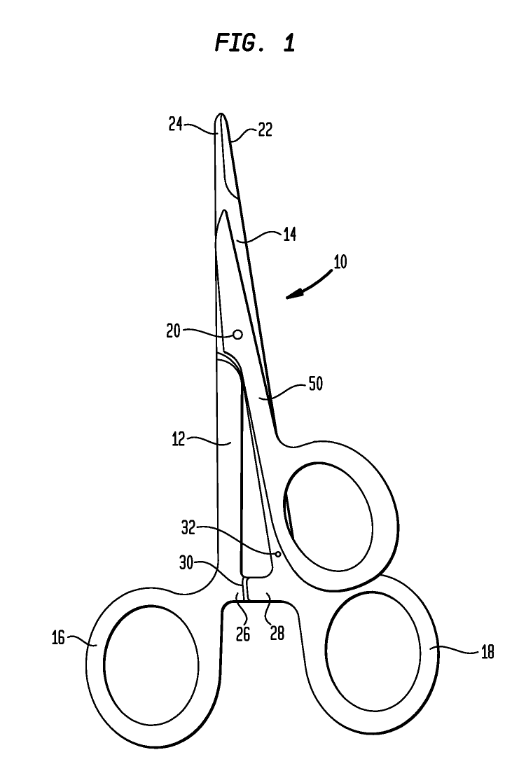

One embodiment of the present invention is a combination surgical needle holder with an attached suture cutter 10 (FIG. 1). The device comprises a pair of elongated arm members 12 and 14, each of which includes a finger ring 16 and 18, respectively, at a first end of the arm (which will be referred to as the proximal end, that is, proximal in relation to the user of the device). The arms are pivotably joined to each other, and to the scissors member 50 by means of a fastener 20 such as a pivot pin, screw or the like. The second end of each arm (referred to as the distal end) terminates in a blunt edged jaw 22 and 24, respectively, and the two arms are joined to each other by means of a pair of interengaging clasps 26. 28 that include a plurality of serrated projections 30 that interlock with one another and clamp the members together.

Arm member 14 includes a projection 32 near the clasp 28, the projection 32 serving as a stop member to limit the movement of the scissors member 50.

The scissors member 50 comprises an elongated body terminating in a finger loop 52 at its' first or proximal end. Fastener 20 passes through opening 54 and pivotably attaches scissors member 50 to the body members 12 and 14. The second or distal end 56 terminates as a front edge 58 which can be blunted, rounded or pointed. The scissors edge 60 is sharpened, and its mating region 62 on body member 12, between the jaw and the opening in body member 12, is also provided with an edge that is sufficiently sharp to cut suture materials. Projection 32 on arm 14 limits the movement of the scissors member 50 with respect to the position of the body member. The blunted front edge reduces the chance of accidentally cutting the patient with the scissors when the suture is being tied or cut. Although not shown in the drawings, it is expressly contemplated that a scissors member having a sharp front edge is within the scope of the present invention.

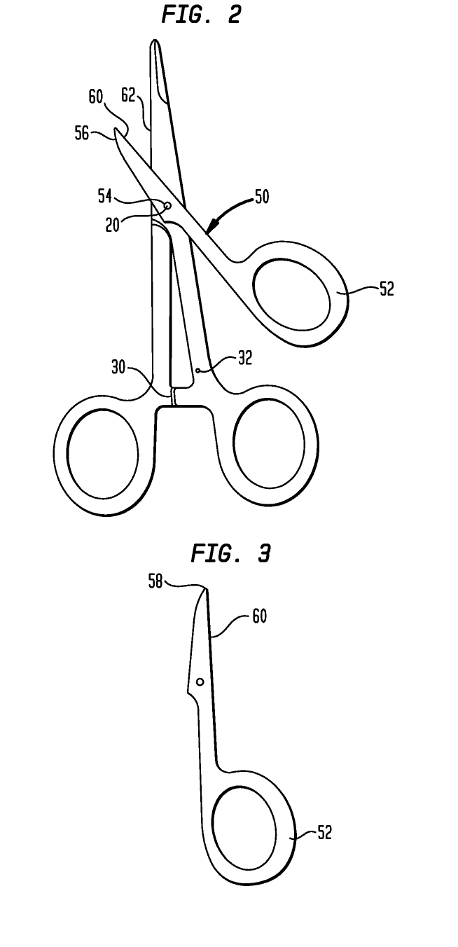

To use embodiments of the present invention, the user grasps the device 10 by putting their thumb and forefinger through the finger rings, release the clasps 26, 28 and separate the body members. A suture needle (not shown) is then gripped between the jaws 22 and 24 of the device, and the device is clamped together using the clasps 26, 28. Clamping of the clamps may be omitted, depending upon the preferences of the individual user. While this step is being performed, the scissors member 50 is maintained in the closed position (see FIG. 1). The user then uses the device to position the suture where desired, and the suture is then tied. Methods of tying sutures are known to those skilled in the art, and further details are thus being omitted. After tying the suture, the user opens the scissors member (FIG. 2) a distance sufficient to separate the scissors member 50 from the arm 14 on which it has been positioned. Generally, the device is opened by moving the scissors member 50 away from where a portion of the finger loop 52 rests against the stop member 32. The device 10 is then positioned such that the suture material is between the scissors cutting edge 60 and arm 14, and the scissors member 50 then brought back to the closed position, resulting in cutting of the suture. The cutting movement of the scissors 50 is limited by the stop 32 positioned on the arm. Using the device 10 with the remaining length of suture material and the needle still within the jaws, the user can place the next suture, tie the suture, cut the suture and repeat the process until the length of suture material has been used, and a new suture length needs to be utilized, or until the opening is closed, as appropriate to the particular situation.

The scissors function is initiated by using the finger loop 52 for the user's index finger. This does not require any additional steps or change in the position of the hand, because the typical position for holding a needle holder is with the thumb and middle fingers in the loops of the needle holder, and the index finger resting adjacent to the middle finger, guiding the middle finger. Using embodiments of the present invention, the index finger is in the same position, but now inside the finger loop 52 of the scissor arm. This instrument can be used by either right or left handed persons by flipping it 180 degrees.

Depending upon the nature and extent of the clinical situation, it may be necessary to apply an anesthetic agent to the site being treated, or to the patient. In the former, the anesthetic may be applied in any form of topical anesthetic, such as a spray-on, injectable, cream, lotion or the like, or for the latter situation, a general anesthetic administered either intravenously, gaseous by a mask, or other appropriate method.

While the examples herein have been directed towards use with human beings, it is to be understood that this instrument can be used with a variety of animal species, ranging from amphibians, fishes, large and small animals typically treated in a veterinary setting or zoological garden, plant species, and in applications as diverse as sewing, needlework, or industrial manufacturing, in situations where a thread or suture or comparable material has to be placed, tied and cut. Some animals are, for example only, and not intended as a limitation, dogs, cats, cows, gorillas, horses, lions, monkeys, sheep, tigers and zebras, to name only a few representative types.

Therefore, although this invention has been described with a certain degree of particularity, it is to be understood that the present disclosure has been made only by way of illustration and that numerous changes in the details of construction and arrangement of parts may be resorted to without departing from the spirit and scope of the invention.

I claim:

1. A surgical instrument, comprising:
a first elongated arm and a second elongated arm;
each arm including a first end and a second end, the first end terminating in a finger ring;
the second end terminating in a jaw, the jaws sized to hold a needle therebetween;
the arms pivotably joined by a fastener; and
a stop member attached to the first arm;
a scissors member, including a first end and a second end, the first end terminating in a finger ring, the scissors member pivotably joined to the arms by the fastener;
the scissors member comprising a cutting edge, the cutting edge positioned between the fastener and the scissors member second end;
the scissors member being attached to the first arm; and
an interengageable clasp attached to each of the arms, the clasp being attached to the arms between the fastener and the finger ring, each of the clasps further comprising a plurality of projections, the projections of the first arm engaging the projections of the second arm when the clasps are engaged, thereby closing the instrument;
whereby a needle is retained between the jaws when the instrument is in a closed position, the needle being attached to a length of suture material, the instrument being useable to place and tie a suture;
the scissors member being opened by using the scissors member finger ring to pivot the scissors member about the fastener, the length of suture material that is not tied being positioned between the scissors member and the first arm of the instrument, and closing the scissors member by using the finger ring to pivot the scissors member about the fastener, thereby cutting the untied length of suture material.

2. The surgical instrument as described in claim 1, wherein the scissors member is attached to the first arm axial to the first arm.

3. The surgical instrument as described in claim 2, wherein the first arm further comprises a lateral edge, the lateral edge being disposed between the fastener and the second end of the arm, the lateral edge being exposed such that the lateral edge opposes the scissors member cutting edge when the scissors member is pivoted about the fastener into the open position.

4. The surgical instrument as described in claim 3, wherein the second end of the scissors member is blunted.

5. The surgical instrument as described in claim 3, wherein the second end of the scissors member is pointed.

6. The surgical instrument as described in claim 3, wherein the stop member is positioned between the finger loop and the fastener.

7. The surgical instrument as described in claim 6, wherein the stop member limits the movement of the scissors member.

8. The surgical instrument as described in claim 3, wherein the scissors member is shorter than either of the arms.

9. A surgical instrument comprising:
a first elongated arm and a second elongated arm;
each arm including a first end and a second end, the first end terminating in a finger ring;
the second end terminating in a jaw, the jaws sized to hold a needle therebetween;
the arms pivotably joined by a fastener; and
a stop member attached to the first arm;
a scissors member, including a first end and a second end, the first end terminating in a finger ring, the scissors member pivotably joined to the arms by the fastener;
the scissors member being shorter than either of the arms;
the scissors member pivotably attached axial to the first arm;
the scissors member comprising a cutting edge, the cutting edge positioned between the fastener and the scissors member second end; and
the second end of the scissors member is blunted;
the first arm further comprising a lateral edge, the lateral edge being disposed between the fastener and the second end of the arm, the lateral edge being opposed to the scissors member cutting edge when the scissors member is moved from being proximate to the stop member to a position away from the stop member, thereby exposing the cutting edge of the scissors member; and
an interengageable clasp attached to each of the arms, the clasp being attached to the arms between the fastener and the finger ring, each of the clasps further comprising a plurality of projections, the projections of the first arm engaging the projections of the second arm when the clasps are engaged, thereby closing the instrument;
whereby a needle is retained between the jaws when the instrument is in a closed position, the needle being attached to a length of suture material, the instrument being useable to place and tie a suture;
the scissors member being opened by using the scissors member finger ring to pivot the scissors member about the fastener, the length of suture material that is not tied being positioned between the scissors member and the first arm of the instrument, and closing the scissors member by using the finger ring to pivot the scissors member about the fastener, thereby cutting the untied length of suture material.

10. A method for suturing, the method comprising the steps of:
placing the suture with a surgical instrument, the instrument comprising:
a first elongated arm and a second elongated arm;
each arm including a first end and a second end, the first end terminating in a finger ring;
the second end terminating in a jaw, the jaws sized to hold a needle therebetween;
the arms pivotably joined by a fastener; and
a stop member attached to the first arm;
a scissors member, including a first end and a second end, the first end terminating in a finger ring, the scissors member pivotably attached to the first arm by the fastener;
the scissors member comprising a cutting edge, the cutting edge positioned between the fastener and the scissors member second end; and
an interengageable clasp attached to each of the arms, the clasp being attached to the arms between the fastener and the finger ring, each of the clasps further comprising a plurality of projections, the projections of the first arm engaging with the projections of the second arm when the clasps are engaged, thereby closing the instrument;
whereby a needle is retained between the jaws when the instrument is in a closed position, the needle being attached to a length of suture material;
tying the suture using the instrument;
opening the scissors member by using the finger ring to pivot the scissors member about the fastener;
placing the length of suture material that is not tied between the scissors member and the first arm of the instrument; and
cutting the untied length of suture material by closing the scissors member by using the finger ring to pivot the scissors member about the fastener.

11. The method as described in claim 10, further comprising the steps of:
placing an additional suture with the instrument;
tying the suture using the instrument;
opening the scissors member by moving the scissors member finger ring away from the stop member;
placing the untied length of suture material between the scissors member and the first arm of the instrument; and
cutting the untied length of suture material by moving the scissors member finger ring proximate to the stop member; and
repeating the steps of placing, tying, opening, placing and cutting until the suture material has all been placed or until the suture material is no longer required.

12. The method as described in claim 10, wherein the scissors member is attached to the first arm axial to the first arm.

13. The method as described in claim 10, wherein the scissors member is shorter than either of the arms.

14. A method for suturing a living organism, the method comprising the steps of:
placing the suture with a surgical instrument, the instrument comprising:
a first elongated arm and a second elongated arm;
each arm including a first end and a second end, the first end terminating in a finger ring;
the second end terminating in a jaw, the jaws sized to hold a needle therebetween;
the arms pivotably joined by a fastener; and
a stop member attached to the first arm;
a scissors member, including a first end and a second end, the first end terminating in a finger ring, the scissors member pivotably attached to the first arm by the fastener;
the scissors member comprising a cutting edge, the cutting edge positioned between the fastener and the scissors member second end; and
an interengageable clasp attached to each of the arms, the clasp being attached to the arms between the fastener and the finger ring, each of the clasps further comprising a plurality of projections, the projections of the first arm engaging the projections of the second arm when the clasps are engaged, thereby closing the instrument;
whereby a needle is retained between the jaws when the instrument is in a closed position, the needle being attached to a length of suture material;
tying the suture using the instrument;
opening the scissors member by using the finger ring to pivot the scissors member about the fastener;
placing the length of suture material that is not tied between the scissors member and the first arm of the instrument; and
cutting the untied length of suture material by closing the scissors member by using the finger ring to pivot the scissors member about the fastener.

15. The method as described in claim 14, wherein the living organism is an animal.

16. The method as described in claim 15, wherein the living organism is a mammal.

17. The method as described in claim 16, wherein the mammal is a human.

18. The method as described in claim 14, further comprising the step of applying an anesthetic to the living organism.

19. A method for suturing, the method comprising the steps of:
placing the suture with a surgical instrument, the instrument comprising:
a first elongated arm and a second elongated arm;
each arm including a first end and a second end, the first end terminating in a finger ring;
the second end terminating in a jaw, the jaws sized to hold a needle therebetween;
the arms pivotably joined by a fastener; and
a stop member attached to the first arm;
a scissors member, including a first end and a second end, the first end terminating in a finger ring, the scissors member pivotably attached to the arms by the fastener;
the scissors members being shorter than either of the arms;
the scissors member pivotably attached axial to the first arm;
the scissors member comprising a cutting edge, the cutting edge positioned between the fastener and the scissors member second end, the scissors member second end being blunted;
the first arm further comprising a lateral edge, the lateral edge being disposed between the fastener and the second end of the arm, the lateral edge being opposite the scissors member cutting edge when the scissors member is moved from being proximate to the stop member to a position away from the stop member, thereby exposing the cutting edge of the scissors member;
an interengageable clasp attached to each of the arms, the clasp being attached to the arms between the fastener and the finger ring, each of the clasps further comprising a plurality of projections, the projections of the first arm engaging the projections of the second arm when the clasps are engaged, thereby closing the instrument; and
whereby a needle is retained between the jaws when the instrument is in a closed position, the needle being attached to a length of suture material;
tying the suture using the instrument;
opening the scissors member by using the finger ring to pivot the scissors member about the fastener;
placing the length of suture material that is not tied between the scissors member and the first arm of the instrument; and
cutting the untied length of suture material by closing the scissors member by using the finger ring to pivot the scissors member about the fastener.
